SEQUENCE LISTING (2 sequences)

SEQ ID NO: 1 - protein, 449 aa, Artificial Sequence
  hu12G8 heavy chain
QVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  hu12G8 light chain
DIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0010224 A1
(43) Pub. Date: Jan. 10, 2019

(54) FORMULATIONS OF ENGINEERED ANTI-IL-10 ANTIBODIES

(71) Applicants: NARASIMHAN Narasimhan, Rahway, NJ (US); Ashwin BASARKAR, Rahway, NJ (US); Soumendu BHATTACHARYA, Rahway, NJ (US); Wendy BENJAMIN, Rahway, NJ (US); Mohammed SHAMEEM, Nanuet, NY (US); Merck Sharp & Dohme Corp., Rahway, NJ (US)

(72) Inventors: Chakravarthy Nachu Narasimhan, Scotch Plains, NJ (US); Ashwin Basarkar, Keasbey, NJ (US); Soumendu Bhattacharya, East Windsor, NJ (US); Wendy Benjamin, North Brunswick, NJ (US); Mohammed Shameem, Nanuet, NY (US)

(73) Assignee: Merck Sharp & Dohme Corp., Rahway, NJ (US)

(21) Appl. No.: 16/064,721

(22) PCT Filed: Dec. 20, 2016
(86) PCT No.: PCT/US2016/067658

§ 371 Date: Jun. 21, 2018

Related U.S. Application Data
(60) Provisional application No. 62/271,120, filed on Dec. 22, 2015.

(51) Int. Cl. C07K 16/24; A61P 35/00; A61K 9/19; A61K 47/12; A61K 47/36; A61K 47/18

(52) U.S. Cl. CPC C07K 16/244; A61P 35/00; A61K 47/183; A61K 47/12; A61K 47/36; A61K 9/19

(57) ABSTRACT

The present invention provides formulations of anti-IL-10 hum12G8, and their use in treating various disorders.

### FIELD OF THE INVENTION

The present invention relates generally to formulations of therapeutic antibodies, and their use in treating various disorders.

### BACKGROUND OF THE INVENTION

Initially known as cytokine synthesis inhibitor factor or CSIF, interleukin-10 (IL-10) is a potent immunomodulator of hematopoietic cells, particularly immune cells. Cells such as activated Th2 cells, B cells, keratinocytes, monocytes and macrophages produce IL-10. See, e.g., Moore et al., Annu. Rev. Immunol. 11:165 (1993). IL-10 inhibits activation and effector functions of a number of cells that include T cells, monocytes and macrophages. In particular, IL-10 inhibits cytokine synthesis, including that of IL-1, IFN-γ, and TNF, by cells such as Th1 cells, natural killer cells, monocytes, and macrophages. See, e.g., Fiorentino et al., J. Exp. Med., 170:2081-2095 (1989); Fiorentino et al., J. Immunol. 146:3444 (1991); Hsu et al., Int. Immunol. 4:563 (1992); Hsu et al., Int. Immunol. 4:563 (1992); D'Andrea et al., J. Exp. Med. 178:1041 (1993); de Waal Malefyt et al., J. Exp. Med. 174:915 (1991); Fiorentino et al., J. Immunol. 147:3815 (1991).

The production of IL-10 in the tumor microenvironment by tumor infiltrating macrophages, dendritic cells, and CD4+ and CD8+ T cells has been shown to inhibit tumor eradication by the immune system (see, e.g., Jarnicki, et al. (2006) J. Immunol. 896-904). Targeting IL-10 anti-tumor activity with an antagonist of IL-10 could provide potent immunostimulatory activity and tumor eradication.

Antibody drugs for use in humans may differ somewhat in the amino acid sequence of their constant domains, or in their framework sequences within the variable domains, but they typically differ most dramatically in the CDR sequences. Even antibodies binding to the same protein, the same polypeptide, or even potentially the same epitope may comprise entirely different CDR sequences. Therapeutic antibodies for use in human beings can also be obtained from human germline antibody sequence or from non-human (e.g. rodent) germline antibody sequences, such as in humanized antibodies, leading to yet further diversity in potential CDR sequences. These sequence differences result in different stabilities in solution and different responsiveness to solution parameters. In addition, small changes in the arrangement of amino acids or changes in one or a few amino acid residues can result in dramatically different antibody stability and susceptibility to sequence-specific degradation pathways. As a consequence, it is not possible at present to predict the solution conditions necessary to optimize antibody stability. Each antibody must be studied individually to determine the optimum solution formulation. Bhambhani et al. (2012) J. Pharm. Sci. 101:1120.

Antibodies are also relatively high molecular weight proteins (˜150,000 Da), for example as compared with other therapeutic proteins such as hormones and cytokines. As a consequence, it is frequently necessary to dose with relatively high weight amounts of antibody drugs to achieve the desired molar concentrations of drug. In addition, it is often desirable to administer antibody drugs subcutaneously, as this enables self-administration. Self-administration avoids the time and expense associated with visits to a medical facility for administration, e.g., intravenously. Subcutaneous delivery is limited by the volume of solution that can be practically delivered at an injection site in a single injection, which is generally about 1 to 1.5 ml. Subcutaneous self-administration is typically accomplished using a pre-filled syringe or autoinjector filled with a liquid solution formulation of the drug, rather than a lyophilized form, to avoid the need for the patient to re-suspend the drug prior to injection. Antibody drugs must be stable during storage to ensure efficacy and consistent dosing, so it is critical that whatever formulation is chosen supports desirable properties, such as high concentration, clarity and acceptable viscosity, and that also maintains these properties and drug efficacy over an acceptably long shelf-life under typical storage conditions.

As a consequence, the need exists for stable formulations of therapeutic antibodies, such as antibodies that bind to human IL-10. Such stable formulations will preferably exhibit stability over months to years under conditions typical for storage of drugs for self-administration, i.e. at refrigerator temperature in a syringe, resulting in a long shelf-life for the corresponding drug product.

### SUMMARY OF THE INVENTION

The present invention provides formulations of humanized anti-IL-10 antibodies, in particular humanized 12G8 (hum12G8) antibody. In particular the present invention provides a formulation comprising an anti-IL-10 antibody, or antigen binding fragment thereof and a histidine or acetate buffer. In certain embodiments, the formulation comprises about 15-50 mg/ml humanized anti-IL-10 antibody hum12G8, or antigen binding fragment thereof; 10 mM histidine buffer, pH 5.5; 0.02% (w/v) (0.2 mg/ml) polysorbate 80; and 7-8% (w/v) (70-80 mg/ml) sucrose, and the antibody comprises a light chain polypeptide comprising the sequence of SEQ ID NO: 2; and a heavy chain polypeptide comprising the sequence of SEQ ID NO: 1. The formulation can be lyophilized for reconstitution or in liquid form.

The present invention also provides a method of treating a proliferative disorder, comprising administering the reconstituted or liquid formulation (solution formulation) to a subject in need thereof. In further embodiments the formulation is used in treating a proliferative disorder. Also contemplated is the use of the solution formulation in the manufacture of a medicament for treating a proliferative disorder.

### BRIEF DESCRIPTION OF THE DRAWINGS

FIG. 1 shows the amino acid sequence of anti-IL-10 hu12G8 light and heavy chain sequences. The CDR regions are underlined.

FIGS. 2A and 2B show Potency by ELISA stability data at 5, 25 and 40° C. storage conditions for 50 mg/ml anti-IL-10 hum 12G8 liquid formulation in 10 mM histidine, 7% (w/v) sucrose, and 0.02% (w/v) PS-80 at pH 5.5. FIG. 2A and 2B represent 9 month and 12 month stability data, respectively.

FIGS. 3A and 3B: Basic variants (%) by HP-IEX stability data at 5, 25 and 40° C. storage conditions for 50 mg/ml anti-IL-10 hum 12G8 liquid formulation in 10 mM histidine, 7% (w/v) sucrose, and 0.02% (w/v) PS-80 at pH 5.5. FIG. 3A and 3B represent 9 month and 12 month stability data, respectively.

FIGS. 4A and 4B: Main peak (%) by UP-SEC stability data at 5, 25 and 40° C. storage conditions for 50 mg/ml anti-IL-10 hum 12G8 liquid formulation in 10 mM histidine, 7% (w/v) sucrose, and 0.02% (w/v) PS-80 at pH 5.5. FIGS. 4A and 4B represent 9 month and 12 month stability data, respectively.

FIG. 5 shows the stability of 15 mg/ml hum12G8 in acetate and histidine based buffer formulations as measured by CE-SDS at 5, 25 and 40° C. storage conditions.

FIG. 6 shows the stability of 15 mg/ml hum12G8 in acetate and histidine based buffer formulations as measured by HP-IEX at 5, 25 and 40° C. storage conditions.

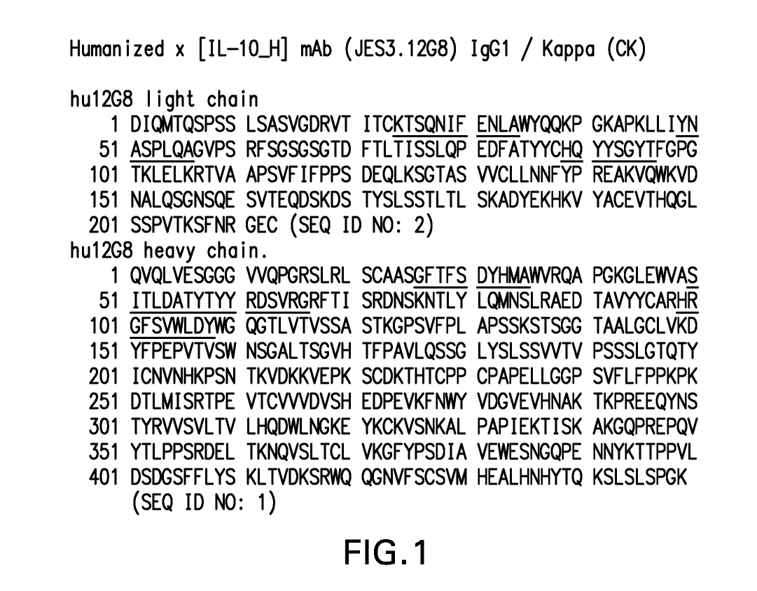

### DETAILED DESCRIPTION

As used herein, including the appended claims, the singular forms of words such as “a,” “an,” and “the,” include their corresponding plural references unless the context clearly dictates otherwise. Unless otherwise indicated, the proteins and subjects referred to herein are human proteins and human subjects, rather than another species. As used herein, “FIG. X” refers collectively to all of individual FIGS. XA-XZ.

### DEFINITIONS

“Proliferative activity” encompasses an activity that promotes, that is necessary for, or that is specifically associated with, e.g. , normal cell division, as well as cancer, tumors, dysplasia, cell transformation, metastasis, and angiogenesis.

The terms “cancer”, “tumor”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer.

As cancerous cells grow and multiply, they form a mass of cancerous tissue, that is a tumor, which invades and destroys normal adjacent tissues. Malignant tumors are cancer. Malignant tumors usually can be removed, but they may grow back. Cells from malignant tumors can invade and damage nearby tissues and organs. Also, cancer cells can break away from a malignant tumor and enter the bloodstream or lymphatic system, which is the way cancer cells spread from the primary tumor (i.e., the original cancer) to form new tumors in other organs. The spread of cancer in the body is called metastasis (What You Need to Know About Cancer—an Overview, NIH Publication No. 00-1566; posted Sep. 26, 2000, updated Sep. 16, 2002 (2002)).

As used herein, the term “solid tumor” refers to an abnormal growth or mass of tissue that usually does not contain cysts or liquid areas. Solid tumors may be benign (not cancerous) or malignant (cancerous). Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

As used herein, the term “primary cancer” refers to the original tumor or the first tumor. Cancer may begin in any organ or tissue of the body. It is usually named for the part of the body or the type of cell in which it originates (Metastatic Cancer: Questions and Answers, Cancer Facts 6.20, National Cancer Institute, reviewed Sep. 1, 2004 (2004)).

As used herein, the term “carcinoma in situ” refers to cancerous cells that are still contained within the tissue where they started to grow, and have not yet become invasive or spread to other parts of the body.

As used herein, the term “carcinomas” refers to cancers of epithelial cells, which are cells that cover the surface of the body, produce hormones, and make up glands. Examples of carcinomas are cancers of the skin, lung, colon, stomach, breast, prostate and thyroid gland.

As used herein, the term “hypervariable region” refers to the amino acid residues of an antibody that are responsible for antigen-binding. The hypervariable region comprises amino acid residues from a “complementarity determining region” or “CDR” (e.g. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain (Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J. Mol. Biol. 196: 901-917). As used herein, the term “framework” or “FR” residues refers to those variable domain residues other than the hypervariable region residues defined herein as CDR residues. The residue numbering above relates to the Kabat numbering system and does not necessarily correspond in detail to the sequence numbering in the accompanying Sequence Listing.

As used herein, concentrations are to be construed as approximate within the ranges normally associated with such concentrations in the manufacture of pharmaceutical formulations. Specifically, concentrations need not be exact, but may differ from the stated concentrations within the tolerances typically expected for drugs manufactured under GMP conditions. Similarly, pH values are approximate within the tolerances typically expected for drugs manufactured under GMP conditions and stored under typical storage conditions. Unless otherwise indicated, percent concentrations are weight/weight concentrations.

### PHARMACEUTICAL COMPOSITION DEFINITIONS

As used herein, an “aqueous” pharmaceutical composition is a composition suitable for pharmaceutical use, wherein the aqueous carrier is distilled water. A composition suitable for pharmaceutical use may be sterile, homogeneous and/or isotonic. Aqueous pharmaceutical compositions may be prepared either directly in an aqueous form, for example in pre-filled syringe ready for use (the “liquid formulations”) or as lyophilisate to be reconstituted shortly before use. As used herein, the term “aqueous pharmaceutical composition” refers to the liquid formulation or reconstituted lyophilized formulation. In certain embodiments, the aqueous pharmaceutical compositions of the invention are suitable for parenteral administration to a human subject. In a specific embodiment, the aqueous pharmaceutical compositions of the invention are suitable for intravenous or subcutaneous administration.

“About” refers to ±10% of the numeric value.

The term “buffer” encompasses those agents which maintain the solution pH in an acceptable range prior to lyophilization and may include succinate (sodium or potassium), histidine, phosphate (sodium or potassium), Tris (tris (hydroxymethyl) aminomethane), diethanolamine, citrate (sodium) and the like. The buffer of this invention has a pH in the range from about 5.0 to about 6.0; and preferably has a pH of about 5.5.

The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

The term “pharmaceutical formulation” refers to preparations which are in such form as to permit the active ingredients to be effective, and which contains no additional components which are toxic to the subjects to which the formulation would be administered.

“Pharmaceutically acceptable” excipients (vehicles, additives) are those which can reasonably be administered to a subject mammal to provide an effective dose of the active ingredient employed.

“Reconstitution time” is the time that is required to rehydrate a lyophilized formulation with a solution to a particle-free clarified solution.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a “stable” liquid antibody formulation is a liquid antibody formulation with no significant changes observed at a refrigerated temperature (2-8° C.) for at least 12 months, preferably 2 years, and more preferably 3 years. In another embodiment, “stable” liquid antibody formulation is a liquid antibody formulation with no significant changes observed at or at room temperature (23-27° C.) for at least 3 months, 6 months, 1 year, 2 years or 3 years. The criteria for stability are as follows: No more than 10%, preferably 5%, of antibody monomer is degraded as measured by SEC-HPLC; the rehydrated solution is colorless, or clear to slightly opalescent by visual analysis, the concentration, pH and osmolality of the formulation have no more than +/−10% change; potency of the antibody is within 60-140%, preferably 80-120% of the control; no more than 10%, preferably 5% of clipping of the antibody is observed; no more than 10%, preferably 5% of aggregation of the antibody is formed.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC) and dynamic light scattering. The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody within 12 months is within 60-140% of the reference. The biological activity of an antibody can be determined, for example, by an antigen binding assay.

The term “isotonic” means that the formulation of interest has essentially the same osmotic pressure as human blood. Isotonic formulations will generally have an osmotic pressure from about 270-328 mOsm. Slightly hypotonic pressure is 250-269 and slightly hypertonic pressure is 328-350 mOsm. Osmotic pressure can be measured, for example, using a vapor pressure or ice-freezing type osmometer.

A “reconstituted” formulation is one that has been prepared by dissolving a lyophilized protein formulation in a diluent such that the protein is dispersed in the reconstituted formulation. The reconstituted formulation is suitable for administration, e.g.

parenteral administration), and may optionally be suitable for subcutaneous administration.

### Analytical Methods

Analytical methods suitable for evaluating the product stability include size exclusion chromatography (SEC), dynamic light scattering test (DLS), differential scanning calorimetery (DSC), iso-asp quantification, potency, UV at 350 nm, UV spectroscopy, and FTIR. SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the .alpha.-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

### IL-10 Antibodies

The CDR residues are highly variable between different antibodies, and may originate from human germline sequences (in the case of fully human antibodies), or from non-human (e.g. rodent) germline sequences. The framework regions can also differ significantly from antibody to antibody. The constant regions will differ depending on whether the selected antibody has a lambda (λ) or kappa (κ) light chain, and depending on the class (or isotype) of the antibody (IgA, IgD, IgE, IgG, or IgM) and subclass (e.g. IgG1, IgG2, IgG3, IgG4).

The IL-10 antibody of the present invention also differs from many recently developed therapeutic antibodies in that it is humanized, rather than fully human. As a result, the CDR sequences are derived from non-human (in this case mouse) germline sequences, rather than human germline sequences. The germline sequences comprise the sequence repertoire from which an antibody's CDR sequences are derived, aside from somatic hypermutation derived changes, and as a consequence it would be expected that CDRs obtained starting with a mouse germline would systematically differ from those starting from a human germline. Use of human germline sequences is often justified on the basis that CDR sequences from human germlines will be less immunogenic in humans than those derived from other species, reflecting the underlying belief that CDRs will systematically differ depending on their species of origin. Although the increase in CDR diversity increases the likelihood of finding antibodies with desired properties, such as high affinity, it further magnifies the difficulties in developing a stable solution formulation of the resulting antibody. In preferred embodiments, the anti-IL-10 antibodies to be used with the claimed formulations are the ones described in U.S. Pat. No. 8,226,947.

Even antibodies that bind to the same antigen can differ dramatically in sequence, and are not necessarily any more closely related in sequence than antibodies to entirely separate antigens. Based on the low sequence similarity, the chemical properties of the antibodies, and thus their susceptibility to degradation, cannot be presumed to be similar despite their shared target.

As discussed above, antibodies are large, highly complex polypeptide complexes subject to various forms of degradation and instability in solution. The diversity of sequence, and thus structure, of antibodies gives rise to wide range of chemical properties. Aside from the obvious sequence-specific differences in antigen binding specificity, antibodies exhibit varying susceptibility to various degradative pathways, aggregation, and precipitation. Amino acid side chains differ in the presence or absence of reactive groups, such as carboxy-(D,E), amino-(K), amide-(N,Q), hydroxyl-(S,T,Y), sulfhydryl-(C), thioether-(M) groups, as well as potentially chemically reactive sites on histidine, phenylalanine and proline residues. Amino acid side chains directly involved in antigen binding interactions are obvious candidates for inactivation by side chain modification, but degradation at other positions can also affect such factors as steric orientation of the CDRs (e.g. changes in framework residues), effector function (e.g. changes in Fc region—see, e.g., Liu et al. (2008) Biochemistry 47:5088), or self-association/aggregation.

Antibodies are subject to any number of potential degradation pathways. Oxidation of methionione residues in antibodies, particularly in CDRs, can be a problem if it disrupts antigen binding. Presta (2005) J. Allergy Clin. Immunol. 116: 731; Lam et al. (1997) J. Pharm. Sci. 86:1250. Other potential degradative pathways include asparagine deamidation (Harris et al. (2001) Chromatogr., B 752:233; Vlasak et al. (2009) Anal. Biochem. 392:145) tryptophan oxidation (Wei et al. (2007) Anal. Chem. 79:2797), cysteinylation (Banks et al. (2008) J. Pharm. Sci. 97:775), glycation (Brady et al. (2007) Anal. Chem. 79:9403), pyroglutamate formation (Yu et al. (2006) J. Pharm. Biomed. Anal. 42:455), disulfide shuffling (Liu et al. (2008) J. Biol. Chem. 283:29266), and hydrolysis (Davagnino et al. (1995) J. Immunol. Methods 185:177). Discussed in Ionescu & Vlasak (2010) Anal. Chem. 82:3198. See also Liu et al. (2008) J. Pharm. Sci. 97:2426. Some potential degradation pathways depend not only on the presence of a specific amino acid residue, but also the surrounding sequence. Deamidation and isoaspartate formation can arise from a spontaneous intramolecular rearrangement of the peptide bond following (C- terminal to) N or D residues, with N-G and D-G sequences being particularly susceptible. Reissner & Aswad (2003) CMLS Cell. Mol. Life Sci. 60:1281.

Antibodies are also subject to sequence-dependent non-enzymatic fragmentation during storage. Vlasak & Ionescu (2011) mAbs 3:253. The presence of reactive side chains, such as D, G, S, T, C or N can result in intramolecular cleavage reactions that sever the polypeptide backbone. Such sequence specific hydrolysis reactions are typically dependent on pH. Id. Antibodies may also undergo sequence-dependent aggregation, for example when CDRs include high numbers of hydrophobic residues. Perchiacca et al. (2012) Prot. Eng. Des. Selection 25:591. Aggregation is particularly problematic for antibodies that need to be formulated at high concentrations for subcutaneous administration, and has even led some to modify the antibody sequence by adding charged residues to increase solubility. Id.

Mirroring the diversity of potential sequence-specific stability issues with antibodies, potential antibody formulations are also diverse. A number of different variables must be custom-optimized for each new antibody. Formulations may vary, for example, in antibody concentration, buffer, pH, presence or absence of surfactant, presence or absence of tonicifying agents (ionic or nonionic), presence or absence of molecular crowding agent. Commercially available therapeutic antibodies are marketed in a wide range of solution formulations, in phosphate buffer (e.g. adalimumab), phosphate/glycine buffer (e.g. basilixumab), Tris buffer (e.g. ipilimumab), histidine (e.g. ustekinumab), sodium citrate (e.g. rituximab); and from pH 4.7 (e.g. certolizumab) and pH 5.2 (e.g. adalimumab) to pH 7.0-7.4 (e.g. cetuximab). They are also available in formulations optionally containing disodium edetate (e.g. alemtuzumab), mannitol (e.g. ipilimumab), sorbitol (e.g. golimumab), sucrose (e.g. ustekinumab), sodium chloride (e.g. rituximab), potassium chloride (e.g. alemtuzumab), and trehalose (e.g. ranibizumab); all with and without polysorbate-80, ranging from 0.001% (e.g. abcixmab) to 0.1% (e.g. adalimumab).

Exemplary antibody formulations are found at U.S. Pat. No. 7,691,379 (anti-IL-9 mAb MEDI-528); U.S. Pat. No. 7,592,004 (anti-IL-2 receptor, daclizumab); U.S. Pat. No. 7,705,132 (anti-EGFR, panitumumab); and U.S. Pat. No. 7,635,473 (anti-Aβ; bapineuzumab). Additional exemplary antibody formulations are found at U.S. Pat. App. Pub. Nos. 2010/00021461 (anti-α4-integrin, natalizumab); 2009/0181027 (anti-IL-12/IL-23, ustekinumab); 2009/0162352 (anti-CD20, ritumixmab); 2009/0060906 (anti-IL-13); 2008/0286270 (anti-RSV, palivizumab); and 2006/0088523 (anti-Her2, pertuzumab). Yet additional formulations are described at Daugherty & Mrsyn (2006) Adv. Drug Deliv. Rev. 58:686; Wang et al. (2007) J. Pharm. Sci. 96:1; and Lam et al. (1997) J. Pharm. Sci. 86:1250.

Sequence variability, which is the basis for antibody specificity, is at the heart of the immune response. This variability leads to chemical heterogeneity of the resulting antibodies, which results in a wide range of potential degradation pathways. The vast array of antibody formulations developed to-date attests to the fact that formulations must be individually optimized for each specific antibody to ensure optimal stability. In fact, each and every commercial therapeutic antibody approved for use in humans so far has had a unique, distinct formulation.

### Biological Activity of Humanized Anti-IL-10

Formulations of the present invention include anti-IL-10 antibodies and fragments thereof that are biologically active when reconstituted. As used herein, the term “biologically active” refers to an antibody or antibody fragment that is capable of binding the desired antigenic epitope and directly or indirectly exerting a biologic effect. Typically, these effects result from the failure of IL-10 to bind its receptor complex. As used herein, the term “specific” refers to the selective binding of the antibody to the target antigen epitope. Antibodies can be tested for specificity of binding by comparing binding to IL-10 to binding to irrelevant antigen or antigen mixture under a given set of conditions.

The solution formulations of anti-IL-10 hum12G8 of the present invention will find use in treatment of disorders in which selective antagonism of IL-10 is expected to be beneficial. Of note, is the combination of IL-10 antibodies with other immunomodulators to treat proliferative disorders such as cancers, and infectious diseases including viral, bacterial, and fungal infections.

The present invention provides formulations of anti-IL-10 hum12G8, which comprises two identical light chains with the sequence of SEQ ID NO: 2 and two identical heavy chains with the sequence of SEQ ID NO: 1, and which is disclosed in U.S. Pat. No. 8,226,947, the disclosure of which is hereby incorporated by reference in its entirety. The humanized light chain 12G8 sequence is provided at SEQ ID NO: 2. The humanized heavy chain 12G8 sequence is provided at SEQ ID NO: 1.

### Solution Formulations

The compositions of this invention minimize the formation of antibody aggregates and particulates and insure that the antibody maintains its bioactivity over time. In one embodiment, the composition is a pharmaceutically acceptable liquid formulation containing a high concentration of an antibody in a buffer having a neutral or slightly acidic pH (pH 5.0-6.5).

In one embodiment, a buffer of pH about 5.0-6.5 or 5.0-6.0 is used in the composition. A buffer of pH 5.5 is preferred. A preferred buffer contains about 10 mM histidine. Sometimes if histidine buffer is used it is overlaid with N₂, to prevent deamidation of the antibody.

A nonionic surfactant polysorbate 80 (Tween® 80) is also added to the formulation. The amount of surfactant added is such that it reduces aggregation of the formulated antibody and/or minimizes the formation of particulates in the formulation and/or reduces adsorption. The surfactant may be present in the formulation in an amount from about 0.005% to about 0.5%, about 0.002% to about 0.04%, preferably from about 0.01% to about 0.1%, more preferably from about 0.01% to about 0.05%, and most preferably from about 0.02% to about 0.04%.

A tonicity modifier, which contributes to the isotonicity of the formulations, can also be added to the solution formulation. The tonicity modifier useful for the present invention includes salts and amino acids. Salts that are pharmaceutically acceptable and suitable for this invention include sodium chloride, sodium succinate, sodium sulfate, potassuim chloride, magnesium chloride, magnesium sulfate, and calcium chloride. In one embodiment, salts are NaCl and MgCl₂. MgCl₂ may also improve the antibody stability by protecting the protein from deamidation. A preferred concentration of NaCl is about 75-150 mM. A preferred concentration of MgCl₂ is about 1-100 mM. Amino acids that are pharmaceutically acceptable and suitable for this invention include proline, alanine, L-arginine, asparagine, L-aspartic acid, glycine, serine, lysine, and histidine.

DTPA and EDTA, which are commonly used chelators to stabilize a protein formulation, may also be included in the formulation. EDTA and DTPA, as chelating agents, may inhibit the metal-catalyzed oxidation of amino acids such as methionine as well as sulfhydryl groups, thus reducing the formation of disulfide-linked aggregates. In addition, antioxidants such as L-methionine can be included in the formulation to prevent oxidative degradation of the antibody.

The liquid antibody formulation of this invention is suitable for parenteral administration such as intravenous, intramuscular, intraperitoneal, or subcutaneous injection; particularly suitable for subcutaneous injection.

### Lyophilized Formulations

Lyophilized formulations of therapeutic proteins provide several advantages. Lyophilized formulations in general offer better chemical stability than solution formulations, and thus increased half-life. A lyophilized formulation may also be reconstituted at different concentrations depending on clinical factors, such as route of administration or dosing. For example, a lyophilized formulation may be reconstituted at a high concentration (i.e. in a small volume) if necessary for subcutaneous administration, or at a lower concentration if administered intravenously. High concentrations may also be necessary if high dosing is required for a particular subject, particularly if administered subcutaneously where injection volume must be minimized.

Typically the lyophilized formulation is prepared in anticipation of reconstitution at high concentration of drug product (DP), i.e. in anticipation of reconstitution in a low volume of liquid. Subsequent dilution with water or isotonic buffer can then readily be used to dilute the DP to a lower concentration. Typically, excipients are included in a lyophilized formulation of the present invention at levels that will result in a roughly isotonic formulation when reconstituted at high DP concentration, e.g. for subcutaneous administration. Reconstitution in a larger volume of water to give a lower DP concentration will necessarily reduce the tonicity of the reconstituted solution, but such reduction may be of little significance in non-subcutaneous, e.g. intravenous administration. If isotonicity is desired at lower DP concentration, the lyophilized powder may be reconstituted in the standard low volume of water and then further diluted with isotonic diluent, such as 0.9% sodium chloride.

The lyophilized formulations of the present invention are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will dictate the time required for drying, which can range from a few hours to several days (e.g. 40-60 hrs). A secondary drying stage may be carried out at about 0-40° C., depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial.

The lyophilized formulations of the present invention are reconstituted prior to administration. The protein may be reconstituted at a concentration of about 10, 15, 20, 25, 30, 40, 50, 60, 75, 80, 90 or 100 mg/mL or higher concentrations such as 150 mg/mL, 200 mg/mL, 250 mg/mL, or 300 mg/mL up to about 500 mg/mL. In other embodiments, the protein concentration after reconstitution is about 10-300, 20-250, 150-250, 180-220, 50-150 or 50 mg/ml. High protein concentrations are particularly useful where subcutaneous delivery of the reconstituted formulation is intended. However, for other routes of administration, such as intravenous administration, lower concentrations of the protein may be desired (e.g. from about 5-50 mg/mL).

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution.

In one embodiment of the present invention, anti-IL-10 antibody (or antigen binding fragment thereof) is formulated as a lyophilized powder for intravenous administration. In another embodiment of the present invention, anti-IL-10 antibody (or antigen binding fragment thereof) is formulated as a lyophilized powder for subcutaneous administration. In certain embodiments, the antibody (or antigen binding fragment thereof) is provided at about 40-300 mg/vial, and is reconstituted with sterile water for injection prior to use. In other embodiments, the antibody (or antigen binding fragment thereof) is provided at about 200 mg/vial, and is reconstituted with sterile water for injection prior to use. In one embodiment, the target pH of the reconstituted formulation is 5.5. In various embodiments, the lyophilized formulation of the present invention enables reconstitution of the anti-IL-10 antibody to high concentrations, such as about 20, 25, 30, 40, 50, 60, 75, 100, 150, 200, 250 or more mg/mL. In other embodiments, the anti-IL-10 antibody concentration after reconstitution is about 10-300, 20-250, 150-250, 180-220, 20-200, 40-100, or 50-150 mg/ml. In other embodiments, the anti-IL-10 antibody concentration pre-lyophilization is about 10-300, 150-250, 180-220, 10-100, 10-50, or 25-50 mg/ml.

The present invention provides in certain embodiments, a lyophilized formulation comprising humanized anti-IL-10 antibody, a histidine buffer at about pH 5.5, at about pH 5.0, at about pH 5.0-6.0, for example at about 5.1, 5.2, 5.3, 5.4, 5.6, 5.7, 5.8, 5.9, or 6.0.

The present invention provides in certain embodiments, a lyophilized formulation comprising humanized anti-IL-10 antibody, an acetate buffer at about pH 5.5, at about pH 5.0, at about pH 5.0-6.0, for example at about 5.1, 5.2, 5.3, 5.4, 5.6, 5.7, 5.8, 5.9, or 6.0.

When a range of pH values is recited, such as “a pH between pH 5.5 and 6.0,” the range is intended to be inclusive of the recited values. Unless otherwise indicated, the pH refers to the pH after reconstitution of the lyophilized formulations of the present invention. The pH is typically measured at 25° C. using standard glass bulb pH meter. As used herein, a solution comprising “histidine buffer at pH X” refers to a solution at pH X and comprising the histidine buffer, i.e. the pH is intended to refer to the pH of the solution.

In other embodiments, the lyophilized formulation of anti-IL-10 antibody, or antigen binding fragment, is defined in terms of the pre-lyophilization solution used to make the lyophilized formulation, such as the pre-lyophilization solution. In one embodiment the pre-lyophilization solution comprises antibody, or antigen-binding fragment thereof, at a concentration of about 10-300, 180-220, 150-250, 45-55 or 50 mg/mL. Such pre-lyophilization solutions may be at about pH 5.5, or range from about pH 5.0 to about 6.0. In a preferred embodiment of the invention, the pre-lyophilized solution comprises: about 45-55 mg/mL of the anti-IL-10 antibody; about 70-80 mg/mL sucrose; about 0.15-0.25 mg/mL polysorbate 80; and about 8-12 mM histidine buffer at about pH 5.0-pH 6.0. In another preferred embodiment of the invention, the pre-lyophilized solution comprises: about 50 mg/mL of the anti-IL-10 antibody, about 70 mg/ml sucrose, about 0.2 mg/ml polysorbate 80, and about 10 mM histidine buffer at pH about 5.5. In another preferred embodiment of the invention, the pre-lyophilized solution comprises: about 200 mg/mL of the anti-IL-10 antibody, about 70 mg/ml sucrose, about 0.2 mg/ml polysorbate 80, and about 10 mM histidine buffer at pH about 5.5.

In yet other embodiments, the lyophilized formulation of anti-IL-10 antibody, or antigen binding fragment, is defined in terms of the reconstituted solution generated from the lyophilized formulation. Reconstituted solutions may comprise antibody, or antigen-binding fragment thereof, at concentrations of about 10, 15, 20, 25, 30, 40, 50, 60, 75, 80, 90 or 100 mg/mL or higher concentrations such as 150 mg/mL, 200 mg/mL, 250 mg/mL, or up to about 300 mg/mL. In one embodiment, the reconstituted formulation may comprise 10-300 mg/mL of the antibody, or antigen-binding fragment thereof. In another embodiment, the reconstituted formulation may comprise 10-60 or 15-50 mg/mL of the antibody, or antigen-binding fragment thereof. In a preferred embodiment, the reconstituted formulation may comprise 45-55 or 50 mg/mL of the antibody, or antigen-binding fragment thereof. Such reconstituted solutions may be at about pH 5.5, or range from about pH 5.0 to about 6.0. In one embodiment of the invention, after reconstitution the formulation comprises: about 10-300 mg/mL of the anti-IL-10 antibody; about 70-80 mg/mL sucrose; about 0.05-0.4 mg/mL polysorbate 80; and about 2-50 mM histidine buffer at about pH 5.0-pH 6.0. In another embodiment of the invention, after reconstitution the formulation comprises: about 10-60 mg/mL of the anti-IL-10 antibody; about 70-80 mg/mL sucrose; about 0.05-0.4 mg/mL polysorbate 80; and about 8-30 mM histidine buffer at about pH 5.0-pH 6.0. In a further embodiment of the invention, after reconstitution the formulation comprises: about 180-220 mg/mL of the anti-IL-10 antibody; about 70-80 mg/mL sucrose; about 0.05-0.4 mg/mL polysorbate 80; and about 8-30 mM histidine buffer at about pH 5.0-pH 6.0. In a preferred embodiment of the invention, after reconstitution the formulation comprises: about 45-55 mg/mL of the anti-IL-10 antibody; about 70-80 mg/mL sucrose; about 0.15-0.25 mg/mL polysorbate 80; and about 8-12 mM histidine buffer at about pH 5.0-pH 6.0. In another preferred embodiment of the invention, after reconstitution the formulation comprises about 150-250 mg/mL of the anti-IL-10 antibody; about 70-80 mg/mL sucrose; about 0.05-0.4 mg/mL polysorbate 80; and about 5-30 mM histidine buffer at about pH 5.0-pH 6.0. In another preferred embodiment of the invention, after reconstitution the formulation comprises about 180-220 mg/mL of the anti-IL-10 antibody; about 70-80 mg/mL sucrose; about 0.15-0.25 mg/mL polysorbate 80; and about 8-12 mM histidine buffer at about pH 5.0-pH 6.0. In yet another preferred embodiment of the invention, the formulation is reconstituted and comprises 50 mg/mL anti-IL-10 antibody, about 7% (w/v) (70 mg/ml) sucrose, about 0.02% (w/v) (0.2 mg/ml) polysorbate 80, and about 10 mM histidine buffer at pH about 5.5.

### Liquid Formulation

A liquid antibody formulation can be made by taking the drug substance which is in for example in an aqueous pharmaceutical formulation and buffer exchanging it into the desired buffer as the last step of the purification process. There is no lyophilization step in this embodiment. The drug substance in the final buffer is concentrated to a desired concentration. Excipients such as sucrose and polysorbate 80 are added to the drug substance and it is diluted using the appropriate buffer to final protein concentration. The final formulated drug substance is filtered using 0.22 μm filters and filled into a final container (e.g. glass vials).

In another aspect of the invention, the anti-IL-10 antibody is in liquid formulation and has the concentration of about 10-300, 20-250, 40-100, 10-60, or 15-50 mg/mL. In another embodiment, the anti-IL-10 antibody is at a concentration of about 15-50 or 10-60 mg/mL. In a preferred embodiment, the anti-IL-10 antibody is at a concentration of about 45-55 or 50 mg/mL. In one embodiment, the liquid formulation comprises a histidine buffer at about pH 5.5, or at about pH 5.0, for example at about 5.0-6.5, 5.0-6.0, 5.1, 5.2, 5.3, 5.4, 5.6, 5.7, 5.8, 5.9, or 6.0. In another embodiment, the liquid formulation comprises an acetate buffer at about pH 5.5, or at about pH 5.0, for example at about 5.0-6.5, 5.0-6.0, 5.1, 5.2, 5.3, 5.4, 5.6, 5.7, 5.8, 5.9, or 6.0.

In other embodiments of the liquid formulation it comprises about 40-100 mg/mL of the anti-IL-10 antibody; about 70-80 mg/mL sucrose; about 0.15-0.25 mg/mL polysorbate 80; and about 2-50 mM histidine buffer at pH about 5.0-6.0. In another embodiment of the liquid formulation it comprises about 10-60 mg/mL of the anti-IL-10 antibody; about 70-80 mg/mL sucrose; about 0.05-0.4 mg/mL polysorbate 80; and about 8-30 mM histidine buffer at pH about 5.0-6.0. In a further embodiment, the liquid formulation comprises about 15-50 mg/mL of the anti-IL-10 antibody.; about 70-80 mg/mL sucrose; about 0.15-0.25 mg/mL polysorbate 80; and about 8-12 mM histidine buffer at about pH 5.0-pH 6.0. In a preferred embodiment, the liquid formulation comprises about 45-55 mg/mL of the anti-IL-10 antibody; about 70-80 mg/mL sucrose; about 0.15-0.25 mg/mL polysorbate 80; and about 8-12 mM histidine buffer at about pH 5.0-pH 6.0. In another preferred embodiment, the liquid formulation comprises about 50 mg/mL of the anti-IL-10 antibody, about 7-8% (w/v) (70-80 mg/ml) sucrose, about 0.02% (w/v) (0.2 mg/ml) polysorbate 80, and about 10 mM histidine buffer at pH about 5.5.

In yet other embodiments, the formulation of anti-IL-10 antibody, is defined in terms of the monomer content of the anti-IL-10 antibody under certain conditions. In one embodiment, at 40° C., the % monomer of the anti-IL-10 antibody is ≥89 or 88% at 12 weeks as measured by size exclusion chromatography. In another embodiment, at 25° C., the % monomer of the anti-IL-10 antibody is ≥95% at 2, 4 or twelve weeks as measured by size exclusion chromatography. In another embodiment, at 5° C., the % monomer of the anti-IL-10 antibody is ≥97.5 or 99% at 2, 4 or twelve weeks as measured by size exclusion chromatography. In a further embodiment, at 5° C., the % monomer of the anti-IL-10 antibody is ≥95% at 6 months as measured by size exclusion chromatography. In yet a further embodiment, at 5° C., the % monomer of the anti-IL-10 antibody is ≥99% at 6 months as measured by size exclusion chromatography. In another embodiment, at 5° C., intact IgG is >=90% at 6 months as measured by Reduced CE-SDS. In yet another embodiment, at 5° C., intact IgG is >=95% at 6 months as measured by Reduced CE-SDS.

In yet other embodiments, the formulation of anti-IL-10 antibody, is defined in terms of the variant content of the anti-IL-10 antibody under certain conditions. In one embodiment, at 40° C., the % basic variant of the anti-IL-10 antibody is ≥10% at 12 weeks or 6 months as measured by ion exchange chromatography. In another embodiment, at 40° C., the % basic variant of the anti-IL-10 antibody is ≥15% at 4 weeks as measured by ion exchange chromatography. In a further embodiment, at 25° C., the % basic variant of the anti-IL-10 antibody is ≥15% at 12 weeks as measured by ion exchange chromatography. In yet another embodiment, at 40° C., the % acidic variant of the anti-IL-10 antibody is less than 68, 69 or 70% at 12 weeks as measured by ion exchange chromatography.

In further embodiments, the liquid formulation comprises about 10-60 mg/mL of the anti-IL-10 antibody; about 70-80 mg/mL sucrose; about 0.05-0.4 mg/mL polysorbate 80; and about 8-30 mM histidine buffer at pH about 5.0-6.0, and at 40° C., the % monomer of the anti-IL-10 antibody is ≥89 or 88% at 12 weeks as measured by size exclusion chromatography. In another embodiment, the liquid formulation comprises about 10-60 mg/mL of the anti-IL-10 antibody; about 70-80 mg/mL sucrose; about 0.05-0.4 mg/mL polysorbate 80; and about 8-30 mM histidine buffer at pH about 5.0-6.0, and at 25° C., the % monomer of the anti-IL-10 antibody is ≥95% at 2, 4 or twelve weeks as measured by size exclusion chromatography.

In further embodiments, the liquid formulation comprises about 10-60 mg/mL of the anti-IL-10 antibody; about 70-80 mg/mL sucrose; about 0.05-0.4 mg/mL polysorbate 80; and about 8-30 mM histidine buffer at pH about 5.0-6.0, and at 40° C., the % basic variant of the anti-IL-10 antibody is ≥10% at 12 weeks as measured by ion exchange chromatography. In another embodiment, the liquid formulation comprises about 10-60 mg/mL of the anti-IL-10 antibody; about 70-80 mg/mL sucrose; about 0.15-0.25 mg/mL polysorbate 80; and about 8-12 mM histidine buffer at pH about 5.0-6.0, and at 40° C., the % basic variant of the anti-IL-10 antibody is ≥10% at 12 weeks or 6 months as measured by ion exchange chromatography.

In yet a further embodiment, the liquid formulation comprises about 10-60 mg/mL of the anti-IL-10 antibody; about 70-80 mg/mL sucrose; about 0.05-0.4 mg/mL polysorbate 80; and about 8-30 mM histidine buffer at pH about 5.0-6.0, and at 40° C., the % acidic variant of the anti-IL-10 antibody is less than 68, 69 or 70% at 12 weeks as measured by ion exchange chromatography. In another embodiment, the liquid formulation comprises about 10-60 mg/mL of the anti-IL-10 antibody; about 70-80 mg/mL sucrose; about 0.15-0.25 mg/mL polysorbate 80; and about 8-12 mM histidine buffer at pH about 5.0-6.0, and at 40° C., the % acidic variant of the anti-IL-10 antibody is less than 68, 69 or 70% at 12 weeks as measured by ion exchange chromatography.

### Dosing and Administration

Various literature references are available to facilitate selection of pharmaceutically acceptable carriers or excipients. See, e.g., Remington's Pharmaceutical Sciences and U.S. Pharmacopeia: National Formulary, Mack Publishing Company, Easton, Pa. (1984); Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (eds.) (1993) Pharmaceutical Dosage Forms: Parenteral Medications, Marcel Dekker, N.Y.; Lieberman, et al. (eds.) (1990) Pharmaceutical Dosage Forms: Tablets, Marcel Dekker, N.Y.; Lieberman et al. (eds.) (1990) Pharmaceutical Dosage Forms: Disperse Systems, Marcel Dekker, N.Y.; Weiner and Kotkoskie (2000) Excipient Toxicity and Safety, Marcel Dekker, Inc., New York, N.Y..

Toxicity is a consideration in selecting the proper dosing of a therapeutic agent, such as a humanized anti-IL-10 antibody (or antigen binding fragment thereof). Toxicity and therapeutic efficacy of the antibody compositions, administered alone or in combination with an immunosuppressive agent, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio of LD50 to ED50. Antibodies exhibiting high therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized.

Suitable routes of administration may, for example, include parenteral delivery, including intramuscular, intradermal, subcutaneous, intramedullary injections, as well as intrathecal, direct intraventricular, intravenous, intraperitoneal. Drugs can be administered in a variety of conventional ways, such as intraperitoneal, parenteral, intraarterial or intravenous injection. Modes of administration in which the volume of solution must be limited (e.g. subcutaneous administration) require that a lyophilized formulation to enable reconstitution at high concentration.

Alternately, one may administer the antibody in a local rather than systemic manner, for example, via injection of the antibody directly into a pathogen-induced lesion characterized by immunopathology, often in a depot or sustained release formulation. Furthermore, one may administer the antibody in a targeted drug delivery system, for example, in a liposome coated with a tissue-specific antibody, targeting, for example, pathogen-induced lesion characterized by immunopathology. The liposomes will be targeted to and taken up selectively by the afflicted tissue.

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002).

Determination of the appropriate dose is made by the clinician, e.g., using parameters or factors known or suspected in the art to affect treatment or predicted to affect treatment. The appropriate dosage (“therapeutically effective amount”) of the protein will depend, for example, on the condition to be treated, the severity and course of the condition, whether the protein is administered for preventive or therapeutic purposes, previous therapy, the patient's clinical history and response to the protein, the type of protein used, and the discretion of the attending physician. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. The protein is suitably administered to the patient at one time or repeatedly. The protein may be administered alone or in conjunction with other drugs or therapies.

Antibodies, or antibody fragments can be provided by continuous infusion, or by doses at intervals of, e.g., one day, 1-7 times per week, one week, two weeks, three weeks, monthly, bimonthly, etc. A preferred dose protocol is one involving the maximal dose or dose frequency that avoids significant undesirable side effects.

In certain embodiments, dosing will comprise administering to a subject escalating doses of 1.0, 3.0, and 10 mg/kg of the pharmaceutical formulation over the course of treatment. Time courses can vary, and can continue as long as desired effects are obtained.

In certain embodiments, the pharmaceutical formulations of the invention will be administered by intravenous (IV) infusion.

In other embodiments, the pharmaceutical formulations of the invention will be administered by subcutaneous administration. Subcutaneous administration may performed by injected using a syringe, or using other injection devices (e.g. the Inject-ease® device); injector pens; or needleless devices (e.g. MediJector and BioJector®).

Subcutaneous administration may be performed by injection using a syringe, an autoinjector, an injector pen or a needleless injection device. Intravenous injection may be performed after diluting the formulation with suitable commercial diluent such as saline solution or 5% dextrose in water.

Although the high concentration solution formulations of the present invention are particularly advantageous for uses requiring a high concentration of antibody, there is no reason that the formulations can't be used at lower concentrations in circumstances where high concentrations are not required or desirable. Lower concentrations of antibody may be useful for low dose subcutaneous administration, or in other modes of administration (such as intravenous administration) where the volume that can be delivered is substantially more than 1 ml. Such lower concentrations can include 60, 50, 40, 30, 25, 20, 15, 10, 5, 2, 1 mg/ml or less.

### Uses

The present invention provides lyophilized or liquid formulations of anti-human IL-10 hum12G8 for use in the treatment of proliferative disorders and conditions, and autoimmune diseases.

The formulations of the present invention can be used in the treatment of, e.g., proliferative disorders such as cancers or tumors, optionally in combination with a TLR9 agonist. Those skilled in the art will realize that the term “cancer” to be the name for diseases in which the body's cells become abnormal and divide without control.

Cancers that may be treated by the compounds, compositions and methods ofthe invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre tumor cervical dysplasia), ovaries (ovarian carcinoma [serous cystadenocarcinoma, mucinous cystadenocarcinoma, unclassified carcinoma], granulosa thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma.

### EXAMPLES

### Example 1

The cDNAs of anti-IL-10 human12G8 (light chain SEQ ID NO:2, heavy chain

SEQ ID NO:1) were codon optimized using the GeneOptimizer® algorithm from GeneArt® (Thermo Fisher Corp.) for expression of the antibody in Chinese hamster ovary cells. The cDNAs for heavy and light chains were synthesized along with Kozak consensus sequence and cloned in two vectors. The expression vector was subsequently used to transfect a CHO cell line. An antibody-expressing clone was selected for the generation of a Master Seed Bank (MSB), based on growth, productivity, and production stability. This MSB was then used to prepare the antibody and to generate the Master Cell Bank (MCB).

Cells from the MCB were expanded in shake flasks, culture bags, and a seed bioreactor to generate the inoculum for a production bioreactor to produce the antibody product. Further processing included three chromatography steps (protein A affinity and anion exchange chromatography), two orthogonal viral clearance steps (low pH viral inactivation and viral filtration), ultrafiltration/diafiltration, and a final 0.2 μm filtration step. The viral filtration product was concentrated by ultrafiltration, and buffer exchange was performed by diafiltering the concentrate with 10 mM histidine pH 5.4. The diafiltered product was then further concentrated to achieve a target anti-IL-10 hum12G8 concentration of 70-80 mg/mL. The final UF product pool was 0.2 μm filtered prior to formulation. The final formulation step entailed the separate additions of the 49% (w/w) sucrose, 10% (w/w) PS-80 stock solutions and 10 mM histidine pH 5.4 to achieve a final drug substance target concentration of 50 mg/mL anti-IL-10 hum12G8 in a 10 mM histidine, 7% (w/v) sucrose, and 0.02% (w/v) PS-80 at pH 5.5.

### Example 2

For the above drug product, 9 month and 12 month stability testing was performed under 5 (ambient humidity), 25 (60% humidity), and 40 (75% relative humidity) ° C. storage conditions.

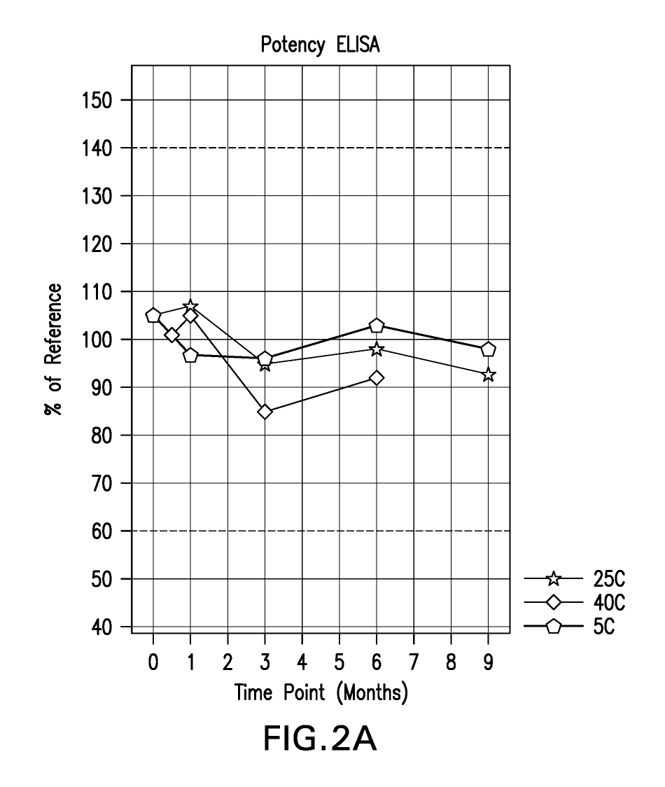

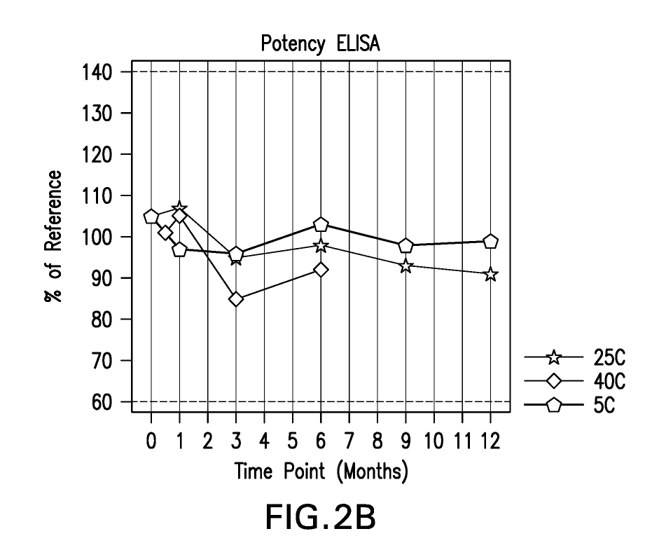

In the ELISA assay, individual dose response curves were generated by using a serial dilution of anti-IL10 reference material or test samples binding to recombinant human IL-10 immobilized on ELISA microtiter plates. Then, by using a SoftMax Pro GxP four-parameter logistic curve fitting analysis, EC₅₀ values of reference material as a control and test samples were determined and a reference-to-sample EC₅₀ ratio was calculated as relative potency for a test sample. Final results were reported as potency relative to reference material. The ELISA results were within the acceptance criteria of 60-140% potency relative to reference set for clinical drug product (FIG. 2).

The samples were analyzed by CE-SDS technique in which protein was denatured with sodium dodecyl sulfate (SDS) under reducing and non-reducing conditions and separated using capillary electrophoresis (CE) (Beckman-Coulter ProteomeLab PA800 CE system and IgG Purity/Heterogeneity Assay Kit). The proteins separate based on their apparent molecular weight. Under non-reducing conditions, all species other than the main IgG peak are classified as impurities. Under reducing conditions, the IgG is resolved into the heavy and light chains. All other species are classified as impurities. In both cases, the result is reported as corrected area percent of each peak as calculated from the total corrected peak area percent.

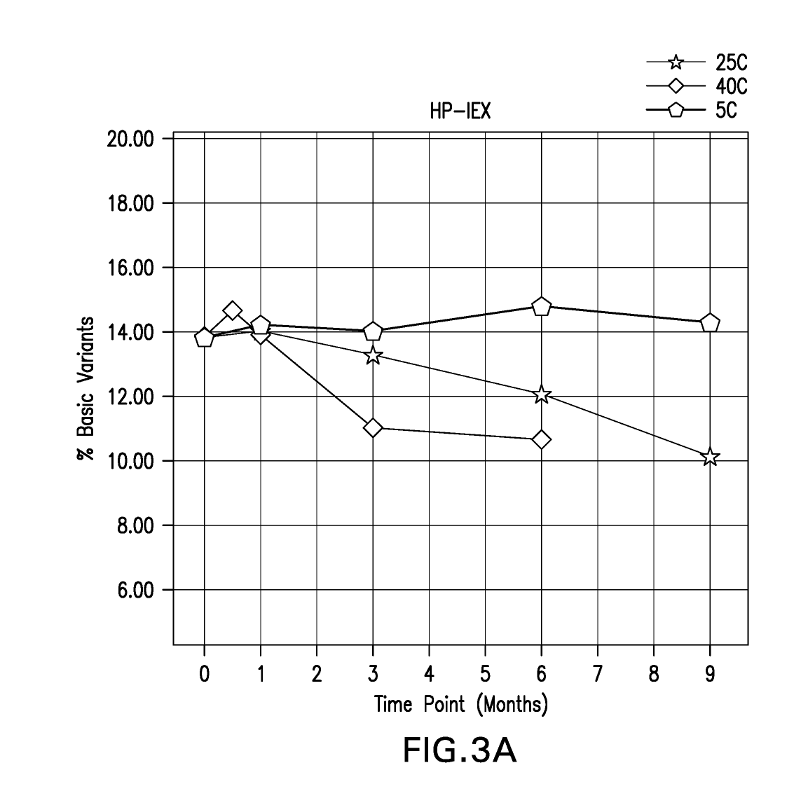

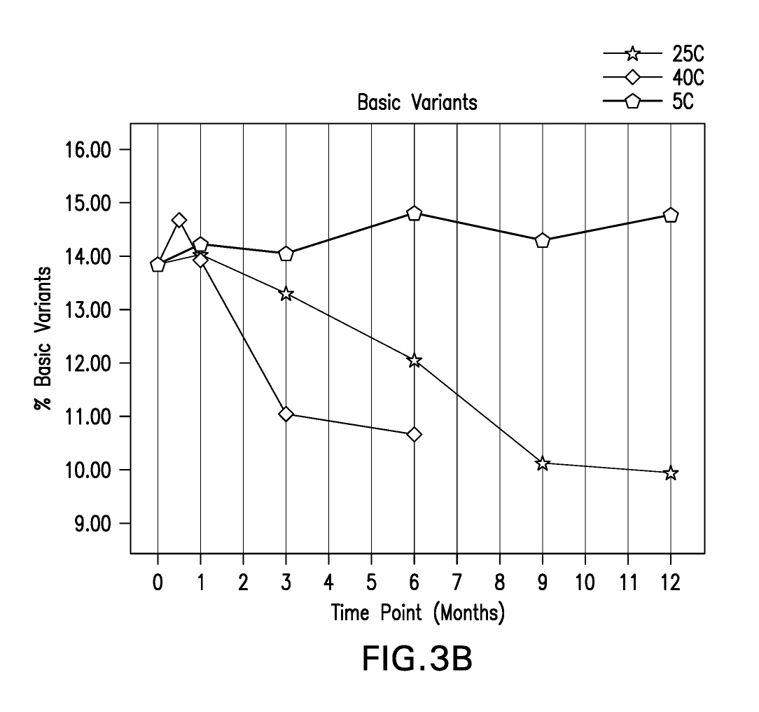

High performance ion-exchange chromatography (HP-IEX) was used to assess purity by revealing the presence of acidic or basic variants. Results are presented as a percentage of total observed material. An ion exchange HPLC method was performed using a Dionex MAbPac SCX-10 column or ThermoFisher Scientific (Dionex) ProPac WCX-10, 4×250 mm column, a UV detector at 280 nm, and mobile phase gradient from 20 mM MOPS, pH 7.2 to 60 mM sodium phosphate, pH 8.0 for elution of bound antibody variants. Characterization of basic variants by HP-IEX for the above drug product is depicted in FIG. 3. Some variability is observed for the basic variants at six months. This is most likely due to assay variability as the value is more in line by the nine month time point and as such can be considered as no notable change overall. At 25° C., a decrease in basic variants from 13.86% to 10.13% was observed. Basic variants drop to 11.06% by three months and drop only slightly further to 10.68% at six months at 40° C.

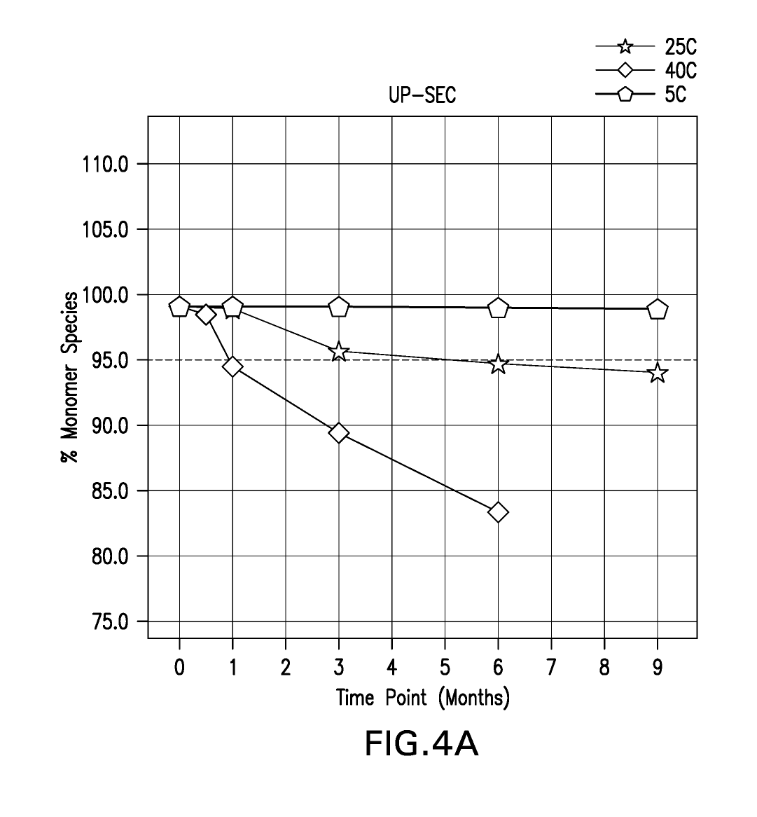

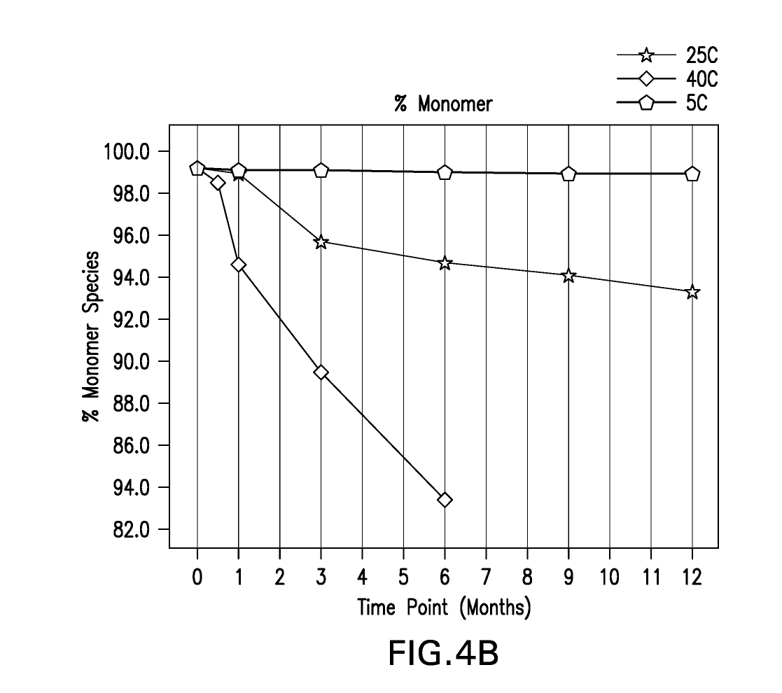

Purity of the sample was further assessed by size exclusion chromatography (SEC) in which the percentage of monomer was determined, as well as the percentages of high molecular weight species (possibly aggregates) and late eluting peaks (possibly degradation products). Size exclusion ultra-high-performance liquid chromatography (SEC-UPLC) was performed by diluting the samples to 1.0 mg/mL in Phosphate Buffered Saline 1×, pH 7.2. Samples were injected into a UPLC equipped with a Waters BEH200 column and a UV detector. Proteins in the sample were separated by size and detected by UV absorption at 214 nm. Characterization of monomer content by UP-SEC for the above formulation is depicted in FIG. 4. A slight decrease in % monomer was observed for the above formulation at 5° C. (from 99.2% to 98.9%) for the 9 month study. There was a decrease in % monomer at 25° C. from 99.2% to 94.1% and a pronounced decrease at 40° C. from 99.2% to 83.4% for the 9 month study.

The turbidity of this Anti-IL-10 hum12G8 histidine formulation increased slightly from 0.111 to 0.117 from the spectrophotometric absorbance at 350 nm at 5° C. at 12 months.

As shown in FIGS. 2B, 3B and 4B, and Tables 7-8, there was no noteworthy change observed for color, degree of opalescence, low molecular weight species, HP-IEX Main and UP-SEC % monomer at 5° C. All stability data at 5° C. for the histidine formulation of Anti-IL-10 hum12G8 were within specifications set for clinical drug product and support a 24-month shelf life.

### Example 3

Anti-IL-10 human12G8 drug substance was dialyzed against 20 mM acetate buffer, pH 5.5 using Millipore centrifugal filter units. Five buffer exchange cycles were performed. Post buffer exchange, protein was recovered and transferred into 50-mL centrifuge tubes and stored at 2-8° C. Concentration of two solutions was measured. Sucrose stock solution (40% w/v) was prepared in 20 mM acetate buffer, pH 5.5. To prepare formulated drug substance, following volumes of stock solutions were used to provide a liquid formulation of anti IL-10 humG8 15 mg/mL, sucrose 8%, polysorbate-80 0.02%, 20 mM acetate buffer, pH 5.5: anti IL-10 humG8 in 20 mM acetate, pH 5.5 (conc. 26.38 mg/ml): 17.06 ml; sucrose stock solution: 6 ml; polysorbate stock solution: 0.6 ml; 20 mM acetate buffer: 6.34 ml. Formulated drug substance was filtered through 0.22 um PVDF membrane using steriflip units. After filtration, each formulation was filled into vials. The vials were stoppered, sealed, and labeled appropriately.

### Example 4

Anti IL-10 12G8 drug substance was dialyzed against 10 mM histidine buffer, pH 5.5 using Millipore centrifugal filter units. Five buffer exchange cycles were performed. Post buffer exchange, protein was recovered and transferred into 50-mL centrifuge tube and stored at 2-8° C. Concentration of two solutions was measured.

Sucrose stock solution (40% w/v) was prepared in 10 mM histidine buffer, pH 5.5. To prepare formulated drug substance, following volumes of stock solutions were used to provide a liquid formulation of anti IL-10 humG8 15 mg/mL, sucrose 8%, polysorbate-80 0.02%, 10 mM histidine buffer, pH 5.5: anti IL-10 in 10 mM histidine, pH 5.5 (conc. 26.97 mg/ml): 16.69 ml; sucrose stock solution: 6 ml; polysorbate stock solution: 0.6 ml; 10 mM histidine buffer: 6.71 ml.

Formulated drug substance was filtered through 0.22 um PVDF membrane using steriflip units. After filtration, each formulation was filled into vials. The vials were stoppered, sealed, and labeled appropriately.

### Example 5

Stability studies for the formulated drug substances prepared in examples 3 and 4 were conducted and analyzed under CE-SDS non-reducing and reducing conditions for purity, HP-IEX for acidic variants, main-peak, basic variants, UP-SEC for high molecular weight species, monomer content and late eluting peaks at up to 12 weeks at 5, 25 and 40° C.

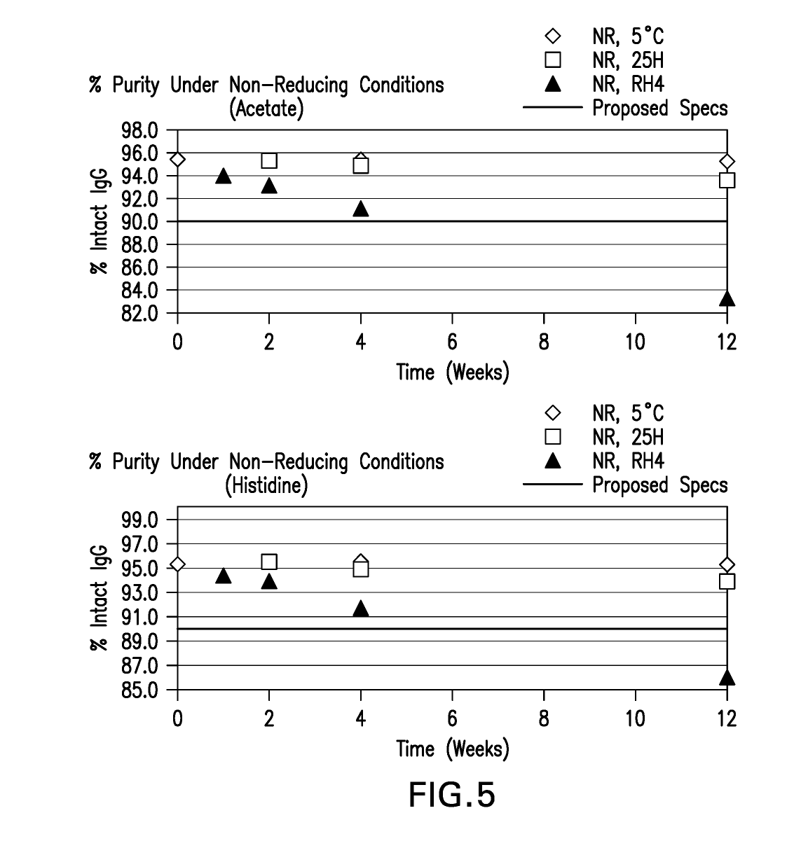

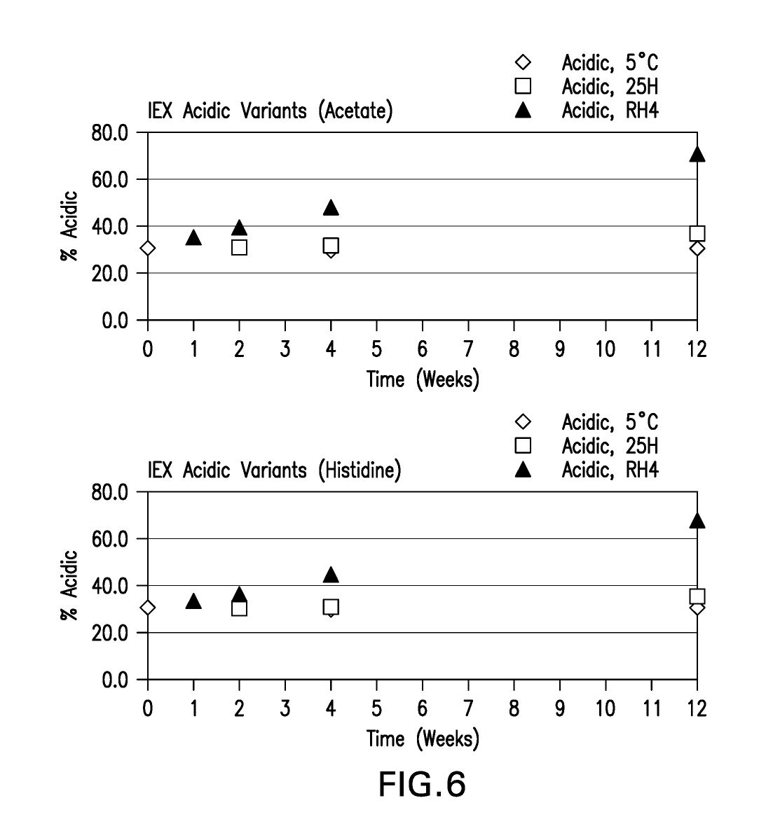

The stability of the samples is illustrated by the various characteristics presented in Tables 1-6 and FIGS. 5 and 6. As compared to the histidine formulation, the UP-SEC data shows that the acetate formulation undergoes higher aggregation and increased fragmentation, and thus more decrease in monomer content over 12 weeks of stability. In addition, HP-IEX data shows more increase in acidic variants for the acetate formulation, with corresponding decrease in basic variants as compared to that of the histidine formulation. This may indicate an increased deamidation of Anti-IL-10 hum12G8 in the acetate formulation over 12 weeks of stability.

TABLE 5

| | Anti-IL-10 Formulation Stability (acetate buffer) | | | | |
|---|---|---|---|---|---|
| | Storage Condition | | | | |
| | 40° C. | | | | |
| | Stability Test Interval | | | | |
| Test | Initial | 1 wk | 2 wk | 4 wk | 12 wk |
| pH | 5.5 | 5.5 | 5.5 | 5.5 | 5.5 |
| Assay - UV | 15.4 mg/mL | 15.2 mg/mL | 15.4 mg/mL | 14.9 mg/mL | 15.1 mg/mL |
| (A280nm) | | | | | |
| Assay - | 0.079 | 0.080 | 0.085 | 0.088 | 0.095 |
| Turbidity | | | | | |
| (A350nm) | | | | | |
| CE-SDS Non- | 95.4% | 94.0% | 93.1% | 91.1% | 83.3% |
| reducing Purity | | | | | |
| CE-SDS | 97.6% | 96.5% | 97.8% | 94.8% | 89.8% |
| Reducing Purity | | | | | |
| HP-IEX | | | | | |
| Acidic Variants | 30.4% | 35.6% | 39.7% | 48.2% | 71.0% |
| Main-Peak | 52.4% | 49.5% | 47.1% | 40.5% | 21.2% |
| Basic Variants | 17.2% | 14.9% | 13.2% | 11.3% | 7.8% |
| UP-SEC | | | | | |
| High Molecular | 0.70% | 0.65% | 0.71% | 0.90%, | 1.29%, |
| Weight Species | | | | 0.09% | 0.32% |
| Monomer | 97.5% | 94.9% | 94.5% | 93.0% | 86.7% |
| Late Eluting | 1.70%, | 3.90%, | 4.06%, | 4.79%, | 8.26%, |
| Peaks | 0.14% | 0.57% | 0.78% | 1.18% | 0.55%, |
| | | | | | 2.88% |

TABLE 6

| | Anti-IL-10 Formulation Stability (histidine buffer) | | | | |
|---|---|---|---|---|---|
| | Storage Condition | | | | |
| | 40° C. | | | | |
| | Stability Test Interval | | | | |
| Test | Initial | 1 wk | 2 wk | 4 wk | 12 wk |
| pH | 5.4 | 5.4 | 5.4 | 5.4 | 5.5 |
| Assay - UV | 15.4 mg/mL | 15.5 mg/mL | 15.4 mg/mL | 15.5 mg/mL | 15.4 mg/mL |
| (A280nm) | | | | | |
| Assay - | 0.073 | 0.079 | 0.088 | 0.106 | 0.185 |
| Turbidity | | | | | |
| (A350nm) | | | | | |
| CE-SDS Non- | 95.3% | 94.4% | 93.9% | 91.7% | 86.0% |
| reducing Purity | | | | | |
| CE-SDS | 97.7% | 96.9% | 96.6% | 95.1% | 89.5% |
| Reducing Purity | | | | | |
| HP-IEX | | | | | |
| Acidic Variants | 30.7% | 33.5% | 36.5% | 44.6% | 67.7% |
| Main-Peak | 52.1% | 49.5% | 46.7% | 39.5% | 21.3% |
| Basic Variants | 17.2% | 17.0% | 16.8% | 15.9% | 11.0% |
| UP-SEC | | | | | |
| High Molecular | 0.67% | 0.39% | 0.45% | 0.91%, | 1.15%, |
| Weight Species | | | | 0.11% | 0.30% |
| Monomer | 97.5% | 95.8% | 95.4% | 93.8% | 89.1% |
| Late Eluting | 1.65%, | 3.33%, | 3.58%, | 4.28%, | 6.64%, |
| Peaks | 0.14% | 0.44% | 0.58% | 0.91% | 0.58%, |
| | | | | | 2.25% |

TABLE 7
Summary of Stability Data for 50 mg/ml anti-IL10 200 mg/vial
Injectable Solution in histidine buffer, 5° C./Amb. RH, Upright

| | Time Point | | | |
|---|---|---|---|---|
| | Initial | 1 month | 3 months | 6 months |
| Potency Binding | 101 | 115 | 99 | 105 |
| ELISA/Biological | | | | |
| Potency, % | | | | |
| HP-IEX, % Acidic | 21.36 | 21.59 | 21.61 | 21.58 |
| Variants | | | | |
| Main | 65.2 | 64.4 | 65.3 | 64.9 |
| Basic Variants | 13.47 | 13.98 | 13.10 | 13.48 |

TABLE 8
Summary of Stability Data for 50 mg/ml anti-IL10 200 mg/vial
Injectable Solution in histidine buffer, 5° C./Amb. RH, Upright

| | Time Point | | | |
|---|---|---|---|---|
| | Initial | 1 month | 3 months | 6 months |
| Purity UP-SEC, % | 99.4 | 991 | 98.9 | 99.0 |
| % Monomer | | | | |
| High Molecular Weight | 0.34 | 0.60 | 0.58 | 0.60 |
| Species | | | | |
| Low Molecular Weight | 0.26 | 0.33 | 0.52 | 0.40 |
| Species | | | | |
| Purity CE-SDS (Non- | 96.9 | 96.8 | 96.9 | 96.8 |
| reduced), % | | | | |
| Purity CE-SDS | 96.1 | 96.1 | 96.1 | 95.7 |
| (Reduced), % | | | | |

1. A liquid formulation comprising: about 10-300 mg/mL of an anti-IL-10 antibody comprising a light chain polypeptide comprising the sequence of SEQ ID NO: 2 and a heavy chain polypeptide comprising the sequence of SEQ ID NO: 1; about 70-80 mg/mL sucrose; about 0.05-0.4 mg/mL polysorbate 80; and about 2-50 mM histidine buffer at pH about 5.0-6.0.

2. The formulation of claim 1, comprising about 10-60 mg/mL of the anti-IL-10 antibody; about 70-80 mg/mL sucrose; about 0.05-0.4 mg/mL polysorbate 80; and about 8-30 mM histidine buffer at about pH 5.0-pH 6.0.

3. The formulation of claim 1, comprising about 40-100 mg/mL anti-IL-10 antibody about 70-80 mg/mL sucrose; about 0.15-0.25 mg/mL polysorbate 80; and about 2-50 mM histidine buffer at pH about 5.0-6.0.

4. The formulation of claim 1, comprising about 15-50 mg/mL of the anti-IL-10 antibody; about 70-80 mg/mL sucrose; about 0.15-0.25 mg/mL polysorbate 80; and about 8-12 mM histidine buffer at about pH 5.0-pH 6.0.

5. The formulation of claim 1 comprising about 45-55 mg/mL of the anti-IL-10 antibody; about 70-80 mg/mL sucrose; about 0.15-0.25 mg/mL polysorbate 80; and about 8-12 mM histidine buffer at about pH 5.0-pH 6.0.

6. The formulation of claim 1, wherein the histidine buffer has a pH of about 5.5.

7. The formulation of claim 1, comprising about 50 mg/mL of the anti-IL-10 antibody; about 70-80 mg/ml sucrose; about 0.2 mg/ml polysorbate 80; and about 10 mM histidine buffer at pH about 5.5.

8. The formulation of claim 1, comprising about 50 mg/mL of the anti-IL-10 antibody; about 70 mg/ml sucrose; about 0.2 mg/ml polysorbate 80; and about 10 mM histidine buffer at pH about 5.5.

9. A lyophilized formulation, wherein after reconstitution, comprises about 10-300 mg/mL of an anti-IL-10 antibody comprising a light chain polypeptide comprising the sequence of SEQ ID NO: 2 and a heavy chain polypeptide comprising the sequence of SEQ ID NO: 1: about 70-80 mg/mL sucrose; about 0.05-0.4 mg/mL polysorbate 80; and about 2-50 mM histidine buffer at pH about 5.0-6.0.

10. The formulation of claim 9, wherein after reconstitution the formulation comprises:
a) about 10-60 mg/mL of the anti-IL-10 antibody;
b) about 70-80 mg/mL sucrose;
c) about 0.05-0.4 mg/mL polysorbate 80; and
d) about 8-30 mM histidine buffer at about pH 5.0-pH 6.0.

11. The formulation of claim 9, wherein after reconstitution the formulation comprises:
a) about 45-55 mg/mL of the anti-IL-10 antibody;
b) about 70-80 mg/mL sucrose;
c) about 0.15-0.25 mg/mL polysorbate 80; and
d) about 8-12 mM histidine buffer at about pH 5.0-pH 6.0.

12. The formulation of claim 9, wherein after reconstitution the formulation comprises:
a) about 150-250 mg/mL of the anti-IL-10 antibody;
b) about 70-80 mg/mL sucrose;
c) about 0.05-0.4 mg/mL polysorbate 80; and
d) about 5-30 mM histidine buffer at about pH 5.0-pH 6.0.

13. The formulation of claim 9, wherein after reconstitution the formulation comprises: about 50 mg/mL of the anti-IL-10 antibody; about 70-80 mg/ml sucrose; about 0.2 mg/ml polysorbate 80; and about 10 mM histidine buffer at pH about 5.5.

14. The formulation of claim 9, wherein after reconstitution the formulation comprises: about 200 mg/mL of the anti-IL-10 antibody; about 70 mg/ml sucrose; about 0.2 mg/ml polysorbate 80; and about 10 mM histidine buffer at pH about 5.5.

15. The formulation of claim 1, wherein at 40° C., the % monomer of the anti-IL-10 antibody is ≥88% at 12 weeks as measured by size exclusion chromatography.

16. The formulation of claim 1, wherein at 40° C., the % acidic variant of the anti-IL-10 antibody is less than 70% at 12 weeks as measured by ion exchange chromatography.

17. The formulation of claim 1, wherein at 5° C., the % monomer of the anti-IL-10 antibody is ≥95% at 6 months as measured by size exclusion chromatography.

18. The formulation of claim 1, wherein at 5° C., the % monomer of the anti-IL-10 antibody is ≥99% at 6 months as measured by size exclusion chromatography.

19. The formulation of claim 1, wherein at 5° C., intact IgG is >=90% at 6 months as measured by Reduced CE-SDS.

20. The formulation of claim 1, wherein at 5° C., intact IgG is >=95% at 6 months as measured by Reduced CE-SDS.

21. A method of treating a proliferative disorder in a subject comprising administering a therapeutically effective amount of the formulation of claim 1 to the subject.
